(19) United States
(12) Patent Application Publication
(10) Pub. No.: US 2015/0306390 A1
(43) Pub. Date: Oct. 29, 2015

(54) METHOD AND APPARATUS FOR SUPPRESSING SEIZURE-LIKE EVENTS

(71) Applicant: NEUROCHIP CORPORATION, Toronto (CA)

(72) Inventors: Osbert C. ZALAY, Toronto (CA); Berj L. BARDAKJIAN, Toronto (CA)

(21) Appl. No.: 14/441,728

(22) PCT Filed: Nov. 8, 2013
(86) PCT No.: PCT/CA2013/000946

§ 371 Date: May 8, 2015

Related U.S. Application Data
(60) Provisional application No. 61/724,897, filed on Nov. 9, 2012.

(51) Int. Cl. A61N 1/36

(52) U.S. Cl. CPC A61N 1/36064

(57) ABSTRACT

An apparatus for synthesizing a multi-band rhythmic signal comprises a therapeutic network having an input to receive an input signal representative of a seizure-like event, said therapeutic network being responsive to said input signal and configured to generate an output stimulation signal of a form to suppress said input signal.

### CROSS-REFERENCE TO RELATED APPLICATIONS

This application claims the benefit of U.S. Provisional Application No. 61/724,897 to Zalay et al. filed on Nov. 9, 2012 and entitled “Method and Apparatus for Suppressing Seizure-Like Events”, the entire content of which is incorporated herein by reference.

### FIELD OF THE INVENTION

The present invention relates to a method and apparatus for suppressing seizure-like events, to a method and apparatus for synthesizing a multi-band rhythmic signal and to a multi-band rhythmic signal.

### BACKGROUND OF THE INVENTION

Neuromodulation by electrical stimulation was initially conceived to treat debilitating pain (Gildenberg 2006). Nowadays, the definition of electrical neuromodulation has expanded to encompass varieties of electrical stimulation targeting motor control, sensory perception and cognition, whereby stimulation is delivered to counter the effects of disability or pathology compromising such functions. For example, functional electrical stimulation (FES) is a technique that modulates nerve activity in the extremities in order to restore lost motor function due to paralysis (Popovic et al. 2011, Thrasher and Popovic 2008, Thrasher et al. 2008). Similarly, pathological motor traits caused by Parkinson's and dystonia have been successfully treated using deep brain stimulation (DBS) (Lozano 2001, Toda et al. 2004), which involves surgical implantation of electrodes delivering electrical pulses to limbic and midline structures of the brain, such as the subthalamic nucleus. DBS has also been favorably indicated for disorders that can alter or otherwise impair cognition, such as epilepsy and depression (Loddenkemper et al. 2001, Mayberg et al. 2005).

For the treatment of epilepsy, most studies involving DBS have implemented open-loop periodic pulse stimulation in which the waveform and frequency of stimulation do not vary over time (Hamani et al. 2009), and whose programmable parameters include amplitude, pulse width, duty cycle and frequency. Recently, however, several groups have turned to investigating closed-loop modes of electrical pulse stimulation. Closed-loop systems require a brain-computer interface (BCI) to record and process data online and to subsequently deliver an appropriately modified stimulus back to the subject. The increased complexity of the stimulator appears to be compensated for by superior performance. In several studies by Osorio and colleagues, patients were administered closed-loop high-frequency stimulation (HFS) (>100 Hz) at the seizure focus, with stimulation being activated automatically when a seizure was detected (Osorio et al. 2001, Osorio et al. 2005, Peters et al. 2001). Patients with bilateral seizure foci had stimulation delivered to the anterior thalamic nucleus (AN). A mean decrease of 55% in the locally-stimulated group (ranging from 100% at best to -36.8% at worst) was achieved, with three (3) of the four (4) patients collectively experiencing an 86% reduction in seizure frequency, whereas the AN stimulated group had a 40.8% mean reduction in seizure occurrence (ranging from 75.6% to −1.4%) (Osorio et al. 2005). In several longitudinal studies, similar benefits were noted in patients who had responsive stimulators implanted for several months or longer (Fountas and Smith 2007, Fountas et al. 2005, Kossoff et al. 2004, Sun et al. 2008).

A study by Wyckhuys and colleagues compared two forms of open-loop (non-responsive) stimulation; namely (1) conventional periodic-pulse HFS, as used in clinical DBS, and (2) Poisson-distributed pulse stimulation (PDS) with the same 130 Hz mean stimulation frequency (Wyckhuys et al. 2010). PDS is biomimetic in the sense that it emulates the observed distribution of neuronal inter-spike intervals, which approximates a Poisson point process (Shadlen and Newsome 1998). Despite the non-responsiveness of PDS, the efficacy of PDS in seizure suppression was significantly improved over that of periodic HFS. In periodic HFS-improved rats, a 50% decrease in seizure occurrence was observed, whereas in PDS-improved rats, seizure frequency declined to 33% of the baseline rate. The only difference between the two forms of stimulation was the complexity of the interval timing of the pulses.

The advantage offered by PDS suggests that biocompatible structuring of the stimulation in terms of signal complexity, waveform and/or rhythmic constituents may be as relevant to neuromodulation performance as the responsiveness of the stimulation.

It is therefore an object to provide a novel method and apparatus for suppressing seizure-like events, a novel method and apparatus for synthesizing a multi-band rhythmic signal and a novel a multi-band rhythmic signal.

### SUMMARY OF THE INVENTION

An approach for synthesizing high-complexity rhythmic or polyrhythmic signals for closed-loop electrical neuromodulation using cognitive rhythm generator (CRG) networks is disclosed herein. In one form, a therapeutic CRG network is used as a rhythmic signal generator to create neuromimetic signals for stimulation purposes. The therapeutic CRG network is interfaced with an epileptiform CRG network that generates spontaneous seizure-like events (SLEs), forming a closed-loop neuromodulation system. SLEs are associated with low-complexity dynamics and a high level of phase coherence in the epileptiform network. The therapeutic CRG network generates a high-complexity, multi-band rhythmic stimulation signal with prominent theta and gamma-frequency power. The stimulation signal suppresses SLEs and increases dynamic complexity in the epileptiform CRG network, as measured by a relative increase in the maximum Lyapunov exponent and decrease in the phase coherence of the epileptiform CRG network output.

Accordingly, in one aspect there is provided an apparatus for synthesizing a multi-band rhythmic signal comprising a therapeutic network having an input to receive an input signal representative of a seizure-like event, said therapeutic network being responsive to said input signal and configured to generate an output stimulation signal of a form to suppress said input signal.

In one form, the therapeutic network comprises an encoding stage in series with a decoding and generating stage. The encoding stage is configured to process and transform the received input signal. The decoding and generating stage in response to encoding stage output generates the output stimulation signal, which can be applied to the system generating the input signal thereby to suppress the seizure-like event. The encoding stage and the decoding and generating stage each comprise a feedback loop. The feedback loop of the decoding and generating stage comprises a time delay. In one embodiment, the encoding stage and the decoding and generating stage each comprise a cognitive rhythm generator (CRG). Each CRG comprises a set or bank of neuronal modes whose mode outputs are combined by mixing functions, a ring device in the form of a limit-cycle oscillator, whose instantaneous amplitude and phase variables are modulated by the combined mode outputs and a mapper that maps the amplitude and phase variables to an observable output variable. In an embodiment, the input signal representative of the seizure-like event is generated by a biologic system. The therapeutic network is configured to apply the stimulation signal to the biologic system to modify the generated input signal.

According to another aspect there is provided a method for suppressing a multi-band rhythmic signal comprising, responsive to an input signal representative of a seizure-like event, generating, using a therapeutic network, an output stimulation signal of a form to suppress said input signal; applying the stimulation signal to the input signal to generate a resultant signal; feeding the resultant signal back to the therapeutic network as said input signal; and repeating said generating, applying and feeding.

In an embodiment, the method further comprises providing a time delay prior to said feeding. The step of generating said output stimulation signal comprises generating at least two rhythm components of different frequency bands, the rhythm component of the lower frequency band modulating or coding the rhythm component of the higher frequency band. In an embodiment, the method further comprises receiving said input signal from a biologic system.

According to another aspect there is provided a multi-band rhythmic signal for suppressing a signal indicative of a seizure-like event comprising at least two rhythm components of different frequency bands, the rhythm component of the lower frequency band modulating or coding the rhythm component of the higher frequency band.

According to yet another aspect there is provided an apparatus for suppressing a multi-band rhythmic signal comprising a therapeutic network having an input configured to receive an input signal representative of a seizure-like event generated by a biologic system, said therapeutic network configured to generate an output stimulation signal of a form to suppress said input signal and apply the stimulation signal to the biologic system to modify the generated input signal, the modified input signal being fedback to the input of said therapeutic network.

### BRIEF DESCRIPTION OF THE DRAWINGS

Embodiments will now be described more fully with reference to the accompanying drawings in which:

FIG. 1 is a schematic diagram of a closed-loop neuromodulation apparatus for synthesizing a multi-band rhythmic signal, the apparatus comprising a therapeutic CRG network;

FIG. 2 includes graphs showing (a) quantification of a seizure-like event (SLE) comprising simulated seizure (S) and non-seizure (NS) epochs by application of a reference threshold (θ=0.2) to the excitation level, E(t), and (b) minimum cost achieved as a function of iteration number for non-gradient parameter optimization by pattern search;

FIG. 3 includes graphs showing CRG neuromodulation stimulus time-frequency characteristics as determined from application of bandpass filtering and wavelet transform;

FIG. 4 includes graphs showing cross-frequency coupling within the CRG neuromodulation stimulus signal, wherein graph (a) is a comparison of time series of theta, gamma and phase-shuffled surrogate gamma, graph (b) is an overlay of normalized amplitudes of the gamma envelope (solid) with the reference theta oscillation (segmented) for model and surrogate gamma time series, graph (c) is an autospectra of the respective model and surrogate gamma signals in graph (a), graph (d) is a mean phase coherence of the envelope of model gamma (solid) and surrogate gamma (segmented) with the theta oscillation, and graph (e) is a modulation index map of gamma frequencies with theta frequencies (left) and theta with delta (right), noting that the color scales are different;

FIG. 5 shows suppression of SLEs in an epileptiform network coupled to the therapeutic CRG network;

FIG. 6 shows a time series distributions of the excitation level function, E(t), and its derivative, E(t), and corresponding empirical cumulative distribution, F(x), where the segmented line is the reference case and the solid line is the stimulated case;

FIG. 7 is a complexity analysis of the epileptiform network time series for the reference case without stimulation (REF) and with the therapeutic CRG network switched on, wherein graph (a) shows short-time maximum Lyapunov exponent values, with the troughs in the REF signal indicating the location of SLEs and graph (b) shows phase coherence time-frequency distribution (left column) and corresponding mean phase coherence time series (right column) with the asterisks denoting the location of SLEs;

FIG. 8 shows complexity measures of different neuromodulation approaches; namely an unstimulated reference

(REF), a 10 Hz low-frequency stimulation (LFS), a 120 Hz high-frequency stimulation (HFS) and closed-loop therapeutic CRG network neuromodulation, wherein graph (a) shows normalized maximum Lyapunov exponent, lower 25th percentile (mean±std. err.) and graph (b) shows normalized mean phase coherence, upper 75th percentile (mean±std. err) with the superscript symbols indicating significance at the 5% level (Wilcoxon rank sum test);

FIG. 9 shows performance measures of therapeutic CRG network neuromodulation with internal and external feedback disrupted: reference closed-loop therapeutic CRG network neuromodulation, internal feedback disrupted (-INT), external feedback disrupted (-EXT), and closed-loop therapeutic CRG network neuromodulation excluding high excitation level runs (CRG(-E)), wherein graph (a) shows percent seizure reduction, graph (b) shows normalized maximum Lyapunov exponent, lower 25th percentile (mean±std. err.) and graph (c) shows normalized mean phase coherence, upper 75th percentile (mean±std. err.) with the superscript symbols indicating significance at the 5% level (Wilcoxon rank sum test); and

FIG. 10 shows spontaneous transition of epileptiform network activity to a higher excitation level during closed-loop therapeutic CRG network neuromodulation, the transition being accompanied by a decline in the MLE values and regularization of spiking dynamics.

### DETAILED DESCRIPTION OF THE EMBODIMENTS

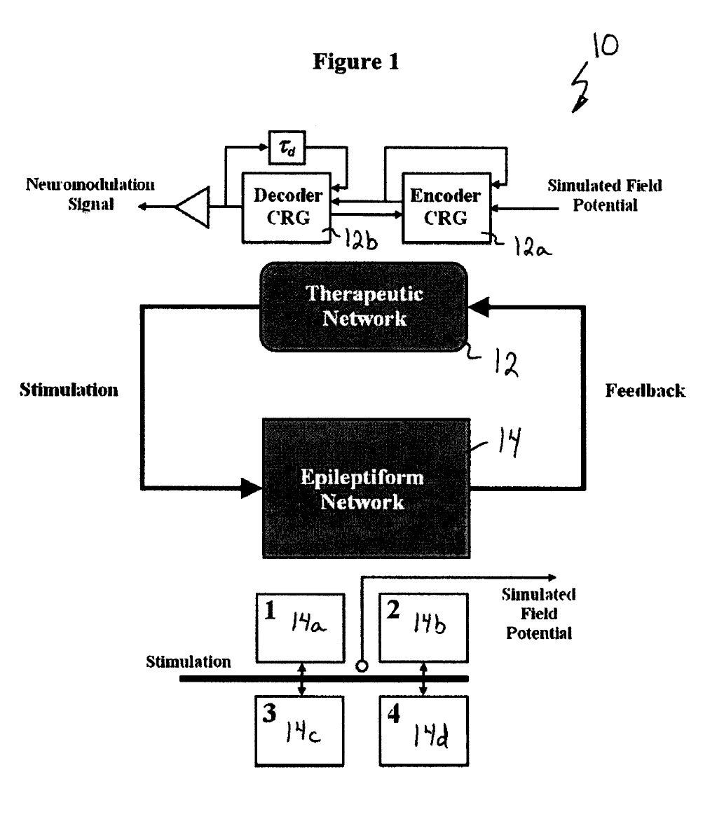

Turning now to FIG. 1, an apparatus for synthesizing high-complexity i.e. multi-band rhythmic or polyrhythmic signals is shown and is generally identified by reference numeral 10. In this embodiment, apparatus 10 comprises a therapeutic network 12 that provides a stimulation signal or neuromodulation to a biological system or network to suppress a signal generated by the biological system that is indicative of a seizure-like event occurring in the biological system. The resultant signal after application of the stimulation signal to the signal generated by the biological system or network is fed back to therapeutic network resulting in closed-loop electrical neuromodulation.

As can be seen, the therapeutic network 12 comprises a plurality of cognitive rhythm generators (CRGs). Each cognitive rhythm generator is of the type described in U.S. Patent Application Publication No. 2010/0292752 to Bardakjian et al. filed on Feb. 17, 2010, the entire disclosure of which is incorporated herein by reference. In this embodiment, the therapeutic network 12 comprises two (2) CRGs 12a and 12b, the minimum number of CRGs for which mutual non-self coupling pathways can exist, enabling the therapeutic network 12 to be computationally efficient and small. Those of skill in the art will however appreciate that larger, higher-order therapeutic CRG networks may be employed to achieve therapeutic efficacy depending on the biological system or network to which the therapeutic network 12 is coupled.

For demonstrative purposes, the therapeutic network 12 was coupled to an epileptiform network 14 configured to generate a signal simulating a spontaneous seizure-like event (SLE). The signal generated by the epileptiform network 14 was applied to the therapeutic network 12, and in response, the therapeutic network 12 generated a stimulation signal of a form to suppress the signal simulating the spontaneous seizure-like event (SLE) that was generated by the epileptiform network 14.

In this embodiment, the epileptiform network 14 comprises a plurality of cognitive rhythm generators (CRGs), in this case, four (4) bi-directionally coupled CRGs 14a to 14d with excitatory-excitatory and excitatory-inhibitory coupling occurring through the integrating mode, m1n. Further specifics of the epileptiform network are described in Zalay et al. 2010, the entire content of which is incorporated herein by reference.

Each cognitive rhythm generator CRG in the epileptiform network 14 and therapeutic network 12 comprises a set or bank of neuronal modes, whose mode outputs are combined by mixing functions, a ring device in the form of a limit-cycle oscillator, whose instantaneous amplitude and phase variables are modulated by the combined mode outputs and a mapper, which constitutes the output static nonlinearity of the CRG and maps the amplitude and phase variables to an observable output variable. The neuronal modes are filters that code for different component input-output dynamics depending on the mode shape and decay profile, and in their most general form are obtained by eigen-decomposition of the Volterra kernels estimated from measurements of the biological system response to input noise. The mode outputs at time t are generated by convolution of the nth CRG input, fₙ(t), with the kth mode, mkₙ (t):

μₖₙ(t)=mₖ_{ₙ}(t)*fₙ(t)=∫₀^{∞}mₖ_{ₙ}(τ)fₙ(t−τ)d τ (1)

For modeling purposes, two modes are utilized, and are selected to have the following analytical expressions:

m₁_{ₙ}(t)=βₙt exp(−βₙt) (2a)

m₂_{ₙ}(t)=βₙ (exp(−βₙt)−m₁_{ₙ}(t)) (2b)

where 1/βₙ is the modal time constant. The convolution operation u₁ₙ(t)=m₁ₙ(t)*fₙ(t) can be represented in equivalent differential form by first taking the Laplace transform such that:

$$\begin{matrix}{U}_{{1}_{n}} ( s ) = {M}_{{1}_{n}} ( s ) {F}_{n} ( s ) \\ = \frac{{β}_{n}}{{( s + {β}_{n} )}^{2}} {F}_{n} ( s ) \\ = \frac{{β}_{n}}{{s}^{2} + 2 {β}_{n} s + {β}_{n}^{2}} {F}_{n} ( s )\end{matrix} \qquad ( 3 )$$

Rearranging Equation (3) and making use of the Laplace transform identity for derivatives, and letting u₁ₙ=\dot{u}₁ₙ(0)=0, the following equivalent second-order differential equation is obtained:

\ddot{μ}₁_{ₙ}+2βₙ\ddot{μ}₁_{ₙ}=βₙfₙ (4)

which upon noting from Equations (2a) and (2b) that m₂ₙ=\dot{m}₁ₙ, can be converted to the following expressions:

\dot{μ}₁_{ₙ}=μ₂_{ₙ} (5a)

\dot{μ}₂_{ₙ}=βₙfₙ−2βₙμ₂_{ₙ}−βₙ²μ₁_{ₙ} (5b)

The form of Equations 5(a) and 5(b) enables the convolutions of input fₙ with the two modes given by Equations (2a) and (2b) to be computed dynamically from the system of first-order differential equations. The mode outputs, u₁ₙ and u₂ₙ, which are the solutions of the equations, dictate how the CRG responds dynamically to coupling inputs or external stimuli through fₙ, which for a network of size M can be written as:

fₙ=Σₘ₌₁^{M}cₘₙyₘ+xₙ (6)

where yₘ is the output of the mth CRG, {cₘₙ} are the associated coupling coefficients, and xₙ is the external input. The mode functions given by Equations (2a) and (2b) have integrating and differentiating character, respectively, in the sense that convolution of mode m₁ₙ with a step input produces an accumulation effect due to its monophasic exponential form. Mode m₂ₙ on the other hand is biphasic and has differentiating character because convolution of the mode with a step input generates a positive output on the rising edge and a negative output on the falling edge, and is everywhere else zero where the input is a constant (Kang et al. 2010, Zalay and Bardakjian 2009). In this way, the mode codes for rate of change of the input, which is akin to taking the derivative.

The mode outputs are combined through mixing the functions to become the inputs to the ring device, which as mentioned above is a limit-cycle oscillator generating the intrinsic dynamics of the CRG, with phase φₙ=arc tan(r₂ₙ/r₁ₙ) modulated by phase mixing function S_{φ,n} and amplitude αₙ=\sqrt{r₁_{ₙ}²+r₂_{ₙ}²} modulated by amplitude mixing function S_{α,n}, according to:

\dot{r}₁_{ₙ}=ωₙ[r₂_{ₙ}(1+S_{φ,n})+r₁_{ₙ}(1+S_{α,n}−r₁_{ₙ}²−r₂_{ₙ}²)] (7a)

\dot{r}₂_{ₙ}=ωₙ[−r₁_{ₙ} (1+S_{φ,n})+r₂_{ₙ}(1+S_{α,n}−r₁_{ₙ}²−r₂_{ₙ}²)] (7b)

The intrinsic angular frequency of the ring device is set by ωₙ. Phase mixing function S_{φ,n} and amplitude mixing function S_{α,n} can be expressed as a linear or nonlinear combination of the mode outputs depending on the modeling requirements. The observable output of the nth CRG is generated by the mapper, a static nonlinearity expressed as:

$${y}_{n} = {c}_{0 , n} + \sqrt{{r}_{{1}_{n}}^{2} + {r}_{{2}_{n}}^{2}} W ( arctan \frac{{r}_{{2}_{n}}}{{r}_{{1}_{n}}} ) + {S}_{y , n} \qquad ( 8 )$$

where W is the intrinsic output waveform normalized to a 2u phase interval, c_{0,n} is a constant offset, and S_{y,n} is an output mixing function dependent on external inputs, mode outputs and internal state variables, specific to the requirements of the model.

The CRG model was implemented computationally in Matlab and Simulink (The MathWorks, Natick, Mass.), utilizing a Gear's method solver of order two (2) for stiff nonlinear differential equations (Gear 1971). Hyperexcitable network conditions are induced by lowering the value of βₙ in Equation (5b) until spontaneous recurrent seizure-like events are observed.

As can be seen in FIG. 1, the therapeutic network 12 comprises three (3) main components, namely a feedback loop to make stimulation responsive, a signal encoding stage to process and transform incoming signals; and a signal decoding and generating stage that feeds an appropriately-rendered, high-complexity biomimetic stimulus back to the biological system or network, in this case simulated by the epileptiform network 14. The external input to the encoding stage in this example is the square of the simulated extracellular field potential of the epileptiform network 14, taken as the scaled sum of the second time-derivatives of the individual CRG outputs of the epileptiform network 14 (Wilson and Bower 1992, Zalay and Bardakjian 2008). The mode outputs are fed to the encoding stage via S_{φ,e} and are mixed linearly according to:

S_{φ,e}=ε_{0,e}+ε_{P,efe}+ε_{1,e}μ_{1,e}+ε_{D,e}μ_{2,e} (9)

where ε₀, ε_{P}, εₗ, and ε_{D} are the constant, proportional, integrating mode and derivative mode mixing coefficients. Similarly, the decoding and generating stage CRG mode outputs are quadratically mixed with the outputs of the encoding stage according to:

S_{φ,d}=S_{φ,e}*[ε_{0,d}+ε_{P,dfd}+ε_{1,d}μ_{1,d}+ε_{D,d}μ_{2,d}] (10)

Nonlinear quadratic mixing allows for frequency-sensitive processing of incoming signals (Zalay and Bardakjian 2009), which contributes to the responsiveness of the neuromodulation. The coupling connections between the encoding stage and decoding and generating stage are bidirectional but also include self-feedback, with the decoding and generating stage incorporating a time delay, r_{d}, in its self-feedback loop to allow for adjustment of the complexity of the stimulus output. A time delay can enable even a simple dynamic system to display complex behavior because the presence of the time delay in the feedback increases the apparent order of the system (Wang et al. 2000). The encoding stage and decoding and generating stage mapper outputs are given by Equation (8), with S_{y,e}=u_{1,e}, S_{y,d}=u_{1,d}, and c_{0,e}=c_{0,d}=0. The stimulation signal generated by the decoding and generating stage and delivered to the epileptiform network 14 via Equation (3) is expressed as:

x_{{epileptiform}}=k_{y,d}y_{d}+k_{1,d}u_{1,d}+k_{D,d}μ_{2,d} (11)

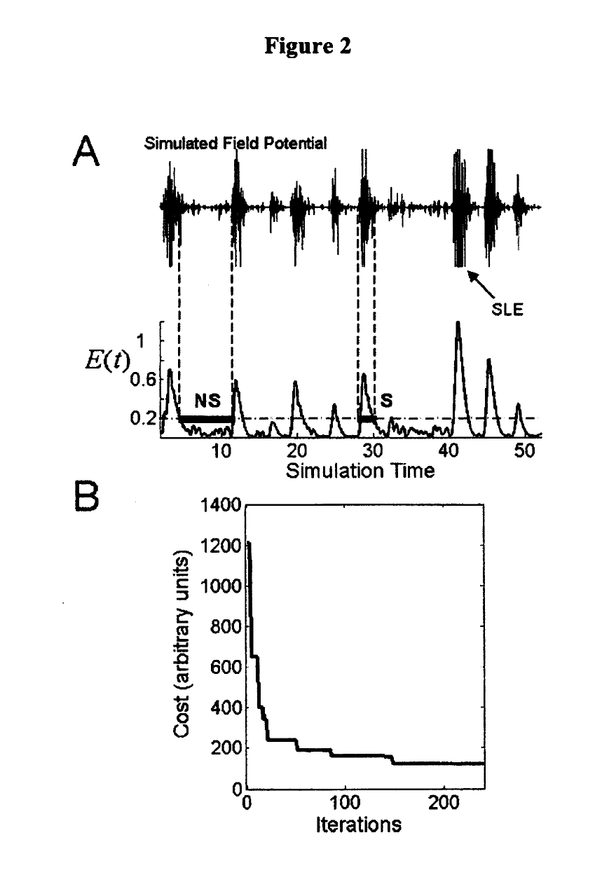

In order to tune the therapeutic network 12, the therapeutic network parameters were determined by performing a series of initial runs of the closed-loop model using randomized parameter values, then selecting a subset of parameter values that yielded promising results with regard to SLE abatement, and finally refining the parameter values through a nonlinear optimization process. To distinguish SLE epochs from non-SLE epochs, a threshold (θ=0.2) was applied to a network excitation level measure (see FIG. 2(a)) which is derived from the state variables reporting the integrating mode (m1ₙ) outputs:

$$E ( t ) = \frac{1}{N} {\sum }_{n = 1}^{N} {[ {u}_{{1}_{n}} ( t ) ]}^{2} \qquad ( 12 )$$

because the integrating mode responses are directly correlated with network excitability (Zalay et al. 2010). The function also correlates with other energy measures, such as the Teager energy operator when applied to the extracellular field potential (Zalay 2011), which makes it feasible to automatically detect SLEs in the biological system from the recorded field potential in a manner analogous to the model.

Nonlinear optimization of therapeutic network parameters (i.e. intrinsic and coupling parameters) was performed using a non-gradient pattern search (Hooke and Jeeves 1961), minimizing a quadratic cost function taking as its arguments (1) the fraction of time spent in seizure, (2) the mean SLE duration, and (3) the neuromodulation stimulus energy. A plot of the lowest error achieved with respect to number of iterations for a successful optimization trial is given in FIG. 2(b). The tuned network parameters are listed in Table 1.

To assess the performance of closed-loop CRG neuromodulation, four (4) quantities were evaluated from the outputs of the epileptiform network 14; namely (1) the percent reduction in SLE time; (2) the percent increase in inter-SLE time; (3) the short-time maximum Lyapunov exponent; and (4) the mean phase coherence. Quantities (1) and (2) pertain to the extent of SLE suppression, whereas quantities (3) and (4) analyze temporal and spatial complexity of the dynamics. These measures were also applied to the results of DBS-like periodic biphasic symmetric rectangular-pulse stimulation of the epileptiform network at low frequency (10 Hz) and high frequency (120 Hz) to compare against the performance of CRG neuromodulation. The formula for percent reduction in SLE time is as follows:

$$Δ {S}_{-} = ( 1 - \frac{T ( > θ )}{{T}_{r} ( > θ )} ) \times 100 \% \qquad ( 13 )$$

where T (>θ) is the time that E(t) is above threshold with neuromodulation switched on, and Tᵣ (>θ) is the reference time above threshold without any stimulation. Likewise the percent increase in non-SLE time is given as a function of time E(t) is below threshold

$$Δ {NS}_{+} = ( \frac{T ( < θ )}{{T}_{r} ( < θ )} - 1 ) \times 100 \% \qquad ( 14 )$$

The maximum Lyapunov exponent (MLE) is a measure of the exponential rate of divergence (+) or convergence (−) of nearby orbits in state space. A positive MLE value for a system exhibiting bounded (Lyapunov stable) dynamics is an indicator of temporal complexity of the dynamics. To numerically compute an estimate of the MLE, the method of time-delay embedding (Packard et al. 1980) was applied to the ring device state variable r₁ₙ to create m-dimensional embedding vectors tracing the dynamical trajectory of the system:

Xᵢ=[r₁_{ₙ}(i)r₁_{ₙ}(i+L) r₁_{ₙ}(i+2L) . . . . r₁_{ₙ}(i+(m−1)L] (15)

with lag L=1 and dimension m=7. The averaged estimate of the MLE over N time segments is therefore given as:

$$λ \approx \frac{1}{NT} {\sum }_{i = 1}^{N} {log}_{2} \frac{‖ δ {X}_{i} ( T ) ‖}{‖ δ {X}_{i} ( 0 ) ‖} \qquad ( 16 )$$

where ∥dXᵢ(0)∥ is the magnitude of the starting separation between test point Xⱼ and the ith reference point Xᵢ on nearby orbits, and ∥dXᵢ(T)∥ is the magnitude of their separation at elapsed time T. The method of computing λ was adapted from the STLmax algorithm for determining the MLE of a short time series (lasemidis et al. 2000). The method does not require computation of the Jacobian of the system, and is not prone to convergence issues or to noise. A sliding-window version of the measure was implemented to accommodate nonstationarity of the time series being evaluated.

The degree of phase coherence between two time series can be obtained by examining the variance of the angular distribution of their phase difference, Δφ(t)=φ₁(t)−φ₂(t) (Hoke et al. 1989, Rosenblum et al. 2001). For complex, nonstationary time series with multiple frequency components or rhythms, the time series can be wavelet-transform decomposed in order to get a time-frequency distribution of phases (Li et al. 2007). With complex wavelet coefficients c_{w}(σₛ, τₙ)+i\tilde{w}(σₛ, τₙ) as a function of scale σₛ and time-shift tₙ and noting that for analytic signals z_{1,2}=x_{1,2}+iy_{1,2} and z₁z₂*=x₁x₂+y₁y₂+i(y₁x₂−x₁y₂), and also that arg(z₁z₂*)=arg(|z₁∥z₂| exp(i(φ₁−φ₂))=Δφ, then:

$$ΔΦ ( {σ}_{s} , {τ}_{n} ) = {tan}^{- 1} \frac{{\tilde{w}}_{1} ( {σ}_{s} , {τ}_{n} ) {w}_{2} ( {σ}_{s} , {τ}_{n} ) - {w}_{1} ( {σ}_{s} , {τ}_{n} ) {\tilde{w}}_{2} ( {σ}_{s} , {τ}_{n} )}{{w}_{1} ( {σ}_{s} , {τ}_{n} ) {w}_{2} ( {σ}_{s} , {τ}_{n} ) + {\tilde{w}}_{1} ( {σ}_{s} , {τ}_{n} ) {\tilde{w}}_{2} ( {σ}_{s} , {τ}_{n} )} \qquad ( 17 )$$

The analysis frequency of a given wavelet at scale σₛ is fₛ=ω_{c}/(2πσₛ). Therefore, averaging over time segment Δτₙ=[τₙ−kT, τₙ+kT], the relative phase coherence (0 to 1) is given by the expression:

$$\begin{matrix}ρ ( {f}_{s} , {Δτ}_{n} ) = \vert 〈 exp ( iΔΦ ( {f}_{s} , {Δτ}_{n} ) ) 〉 \vert \\ = {( \begin{matrix}{( [ \frac{1}{( 2 k + 1 )} {\sum }_{b = - k}^{k} cos ( ΔΦ ( {f}_{s} , {τ}_{n} + bT ) ) ] )}^{2} + \\ {( [ \frac{1}{( 2 k + 1 )} {\sum }_{b = - k}^{k} sin ( ΔΦ ( {f}_{s} , {τ}_{n} + bT ) ) ] )}^{2}\end{matrix} )}^{1 / 2}\end{matrix} \qquad ( 18 )$$

so that by computing the values of Equation (18) over M scales and N shifts, the time-frequency distribution of phase coherence is obtained. The mean phase coherence averaged across frequency bands can then be written as:

$$\overline{ρ} ( {Δτ}_{n} ) = \frac{1}{M} {\sum }_{s = 1}^{M} ρ ( {f}_{s} , {Δτ}_{n} ) \qquad ( 19 )$$

The mean phase coherence measure was applied to the outputs of CRG 14a and CRG 14b of the epileptiform network 14, and to frequency bands extracted from the CRG neuromodulation stimulus.

To assess the properties of the CRG neuromodulation stimulus, some of the same measures used to evaluate neuromodulation performance were also applied to the stimulus signal. Dynamic complexity was quantified by the maximum Lyapunov exponent estimate from Equation (16), as well as by the correlation dimension:

$$v = {lim}_{ξ \to 0} \frac{ln C ( ξ )}{ln ξ} \qquad ( 20 )$$

computed from an adaptation of the algorithm proposed by Grassberger and Procaccia (Grassberger and Procaccia 1983, Theiler 1987, 1990). The correlation integral is estimated from the sum:

$$C ( ξ ) \approx \frac{2}{N ( N - 1 )} {\sum }_{i < j}^{N} Θ ( ξ - ‖ {X}_{i} - {X}_{j} ‖ ) \qquad ( 21 )$$

where θ(·) is the Heaviside function and {Xᵢ}_{i∈N} constitute a series of points on the attractor obtained by time-delay embedding.

Cross-frequency relationships between characteristic frequency bands within the stimulus signal were quantified by calculating the mean phase coherence ((Equation 19)) of the envelope of the higher-frequency signal with the amplitude of the lower-frequency signal, and secondly, by computing the modulation index (Canolty et al. 2006):

$$u ( {f}_{L} , {f}_{H} ) = \frac{\vert \overline{z} ( {f}_{L} , {f}_{H} ) \vert - \overline{\vert {\overline{z}}_{Σ} ( {f}_{L} , {f}_{H} , τ ) \vert}}{{σ}_{Σ} ( {f}_{L} , {f}_{H} , τ )} \qquad ( 22 )$$

which measures cross-frequency coupling between the phase, φ_{L}, of a signal at lower frequency, f_{L}, and the amplitude, φ_{H}, of a signal at higher frequency, f_{H}. The amplitude and phase were obtained from the magnitude and angle of the complex wavelet coefficients. Overhead bars in Equation (22) represent the mean of the quantity underneath, z is a composite complex-valued analytic signal: z(t)=α_{H}(t)exp(iφ_{L}(t)), where z_{Σ} is a surrogate of z created by time-shifting α_{H} relative to φ_{L}, z(t, τ)=α_{H}(t+τ)exp(iφ_{L}(t)), and σ_{Σ} is the standard deviation of the distribution of moduli of time-shifted surrogate means, | z̅_{Σ}(τ)|. Therefore, the normalized deviation of the modulus of z̅ from the mean of the distribution of | z̅_{Σ}(τ)| gives the relative cross-frequency coupling strength.

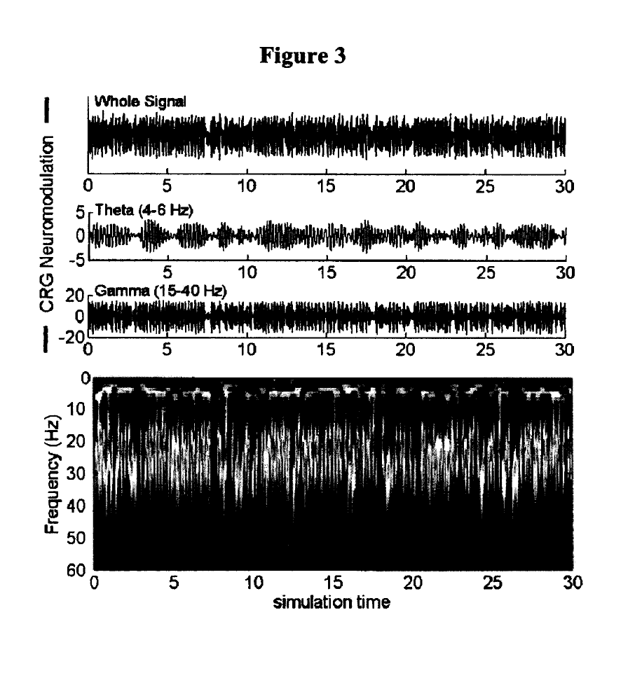

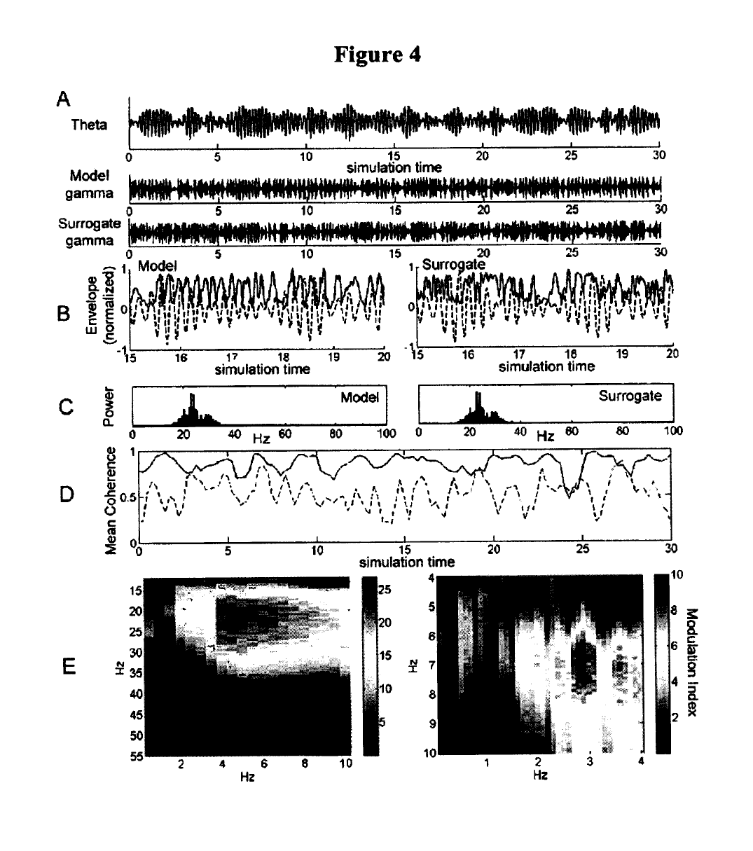

Utilizing the parameterization given in Table 1, the output of the tuned therapeutic network 12 in closed-loop operation generated a rhythmic signal that resembles an extracellular field potential trace (see FIG. 3). A plot of the time-frequency spectrum derived from the wavelet scalogram reveals two predominant rhythmic components with power concentrated in the 4 to 6 Hz and 15 to 40 Hz bands, which roughly correspond to theta and gamma frequency ranges in biological recordings of neural activity. Further investigation of the relationship between the theta and gamma bands revealed that the envelope of gamma (e_{γ}(t)={square root over ([s_{γ}(t)]²+[H(s_{γ}(t))]²)}, where H(s_{γ}(t)) is the Hilbert transform of the gamma band signal, S_{y}) is phase-cohered to the theta cycles, indicating that the two bands are dynamically coupled (see FIG. 4). As will be appreciated, the rhythmic component at the lower frequency (i.e. the slower rhythm) modulates or codes the rhythmic component at the higher frequency (i.e. the faster rhythm).

By contrast, a surrogate gamma signal generated by shuffling the phase while preserving the spectral characteristics of the original signal (Schreiber and Schmitz 2000) yielded lower values of mean coherence of its envelope with theta. The results from the model are suggestive of the theta-gamma cross-frequency coupling that occurs in biological neural network oscillations (Belluscio et al. 2012). These results were corroborated by computing the modulation index over frequency ranges spanned by the stimulus (FIG. 4(e)). Besides confirming the dominant theta-gamma cross-frequency coupling, the theta range was found to be modulated at delta frequencies (<4 Hz), although the modulation was much weaker in magnitude than for theta-gamma.

The temporal complexity and the dimensionality of the neuromodulation stimulus are likewise comparable to biologically recorded brain signals, which are noted in literature to possess positive MLEs (Babloyantz and Destexhe 1986, Chiu et al. 2006, Fell et al. 1993, lasemidis et al. 2000) and correlation dimensions between 2 and 10 (Coenen 1998, Serletis et al. 2011). In particular, bandlimited theta and gamma rhythms extracted from the human awake EEG display maximum Lyapunov exponent values of 2-4 bit/sec and correlation dimension values of 4-6 (Jing and Takigawa 2000, Roschke et al. 1997), comparable to values generated by the therapeutic network 12 (see Table 2).

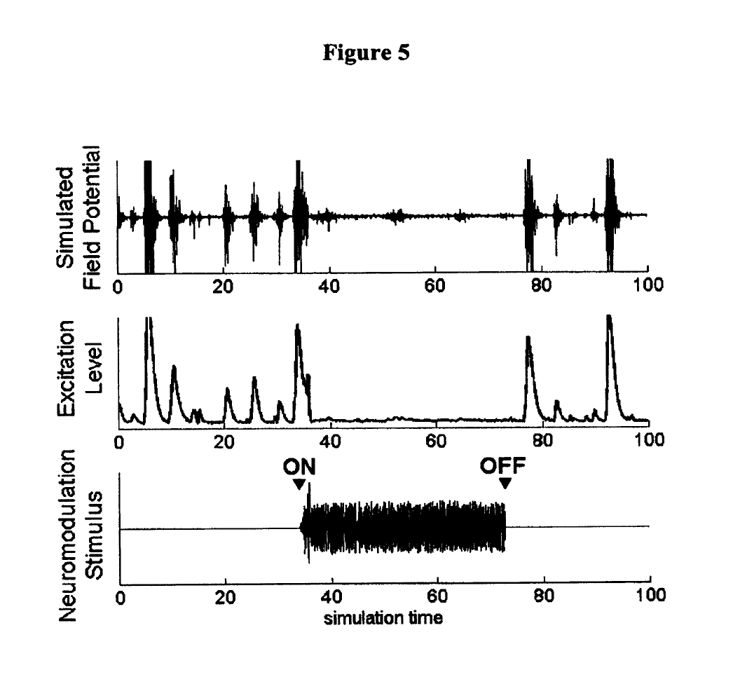

To determine the efficacy of SLE suppression of the low-frequency periodic, high-frequency periodic and CRG neuromodulation paradigms, multiple simulated epochs (N˜120) of time duration T=90 were run using different randomized initial conditions. The excitation level of the unstimulated reference and the neuromodulation-active case were both thresholded to identify SLE and non-SLE regions and to allow quantification of the differences between the two cases for each of the runs. Overall, closed-loop CRG neuromodulation was superior to both 120 Hz HFS and 10 Hz LFS in terms of SLE suppression and improvement (lengthening) of non-SLE times (see Table 3). For neuromodulation-improved runs (i.e. SLE reduction percentage greater than zero), both LFS and HFS were comparable in performance, averaging around 35% SLE suppression rate, whereas CRG neuromodulation achieved a suppression rate of 90%. However, only 46% of LFS runs showed improvement with stimulation, whereas HFS-improved runs accounted for 64% of the total, and CRG neuromodulation-improved runs accounted for nearly 93% of all runs. Taking into consideration both improved runs and stimulation-worsened runs, LFS performed the worst (−2.7%), HFS was in the middle (6.4%), and CRG neuromodulation was still a respectable 82.6%. Non-seizure times were improved the most for CRG neuromodulation and improved the least for LFS overall. FIG. 5 depicts an example simulation run demonstrating SLE suppression for the CRG neuromodulation paradigm.

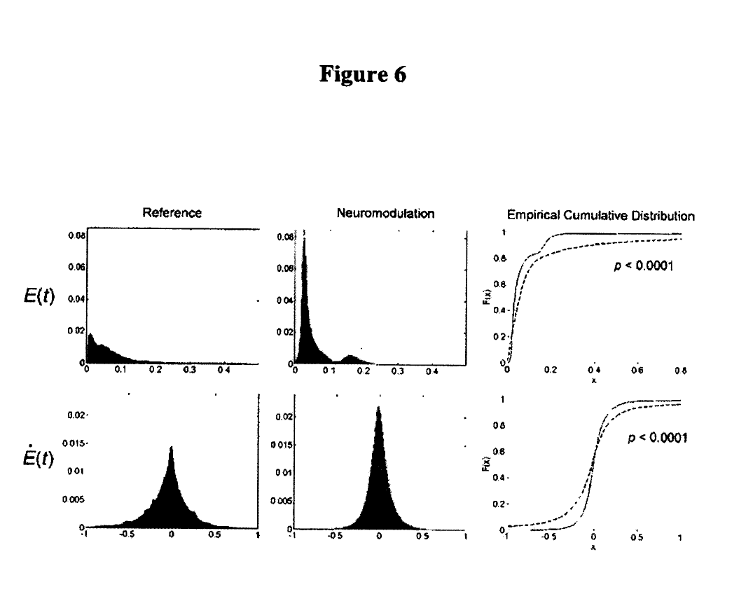

The high-complexity rhythmic stimulation delivered to the epileptiform network 14 effectively paces the activity of that network, maintaining the network in a dynamic state that is removed from the epileptiform reference state which produces seizure-like activity. This is made evident by comparing the reference and stimulation cases in terms of the distribution of values of the excitation level function, E(t), and its derivative, E(t) (see FIG. 6), which are proportional to {μ₁ₙ(t)}² and {u₁ₙ(t)u₂ₙ(t)} from the state variables of the model, respectively. The two-sample Kolmogorov-Smirnov test for equality of distributions was applied to the entire set of simulation runs (Young 1977), revealing that the time series distributions for the reference and stimulated cases differ significantly (p<0.0001), indicating the two conditions are associated with different dynamical states. Neuromodulation had the effect of reducing the spread of E(t), corresponding to more time spent by the network at low excitation values, which is characterized by the narrower, higher peak at small E(t). The stimulated distribution is also slightly bimodal, showing a miniature secondary peak between 0.1 and 0.2, suggesting the presence of another attractor in the dynamics at slightly higher excitation values. The empirical cumulative distribution, F(x), representing the fraction of values in the time series that are below a given x (the Kolmogorov-Smirnov test statistic is defined by supremum of the absolute difference between F(x) of the two sample distributions), illustrates that the stimulated case converges much faster to unity than the reference case for increasing x, so that the tail of the time series distribution falls off much quicker in the stimulated case than the reference case, confirming the result that the stimulated condition is associated with lower values of excitation. Neuromodulation also had the effect of normalizing the values of Ė(t), reducing the abruptness of fluctuations in excitation associated with the epileptiform state.

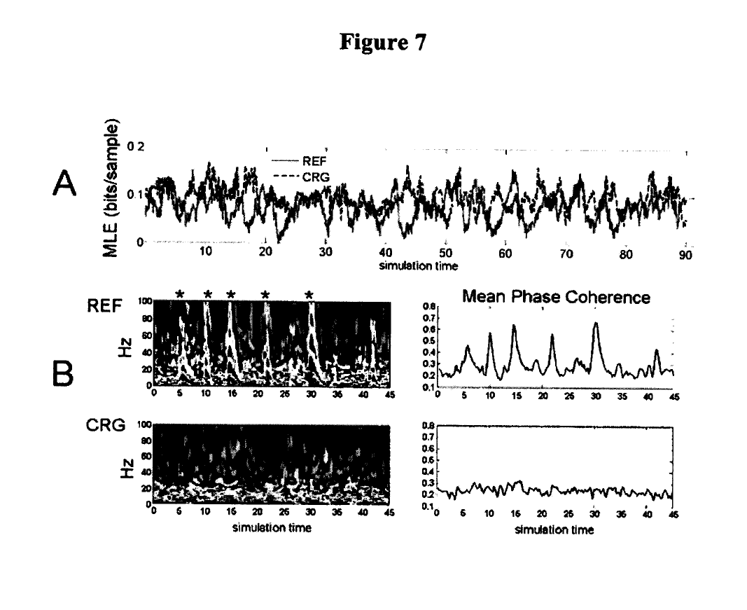

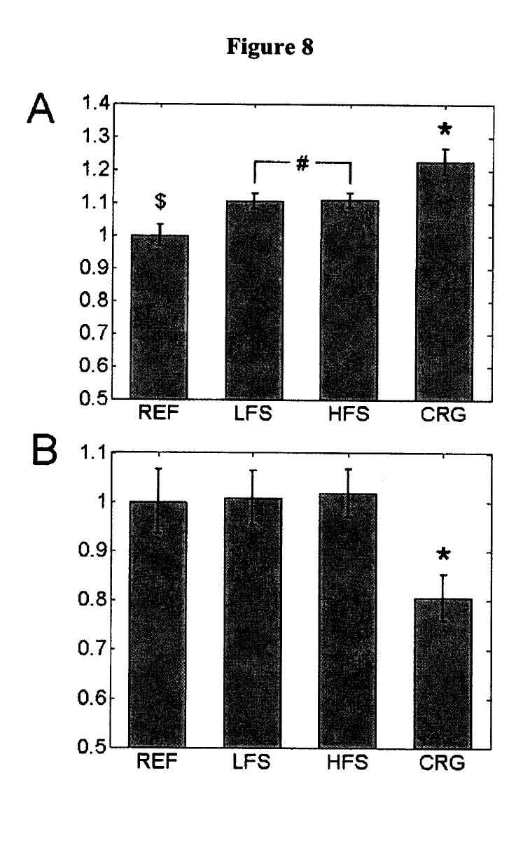

Epileptiform activity is characterized by lower temporal complexity and higher synchrony, as compared to healthy or interictal brain activity. The lower complexity is quantified by a decrease in the maximum Lyapunov exponent (Bergey and Franaszczuk 2001, Chiu et al. 2006, Nair et al. 2009) and the higher synchrony by an increase in phase coherence (Li et al. 2007, Mormann et al. 2000). One of the prescribed goals of neuromodulation is to boost temporal and spatial complexity, which should result in the opposite change (i.e. increased MLE and decreased coherence). The short-time MLE measure based on Equation (16) and the mean phase coherence (Equation (19)) were calculated for each of the 120 runs described above. The results are plotted in FIG. 7 and FIG. 8. The bar plots represent the lower 25th-percentile values of the MLE and the upper 75th-percentile values of the mean phase coherence, respectively, since the objective is to raise the lowest values of the MLE and reduce the highest values of coherence associated with pathological dynamics. The values are normalized to the reference condition (i.e. no stimulation). As expected, the reference condition had the lowest complexity. For periodic biphasic-pulse stimulation, HFS marginally outperformed LFS, although the difference was not statistically significant. Interestingly, despite the large gap in frequency between HFS and LFS and the disparity in overall suppression performance, the resultant complexity from stimulation with either paradigm was nearly equivalent. Furthermore, periodic stimulation had a tendency to further entrain the network, resulting in higher rather than lower coherence values. CRG neuromodulation had the highest MLE values and the lowest coherence, which were significantly improved over periodic stimulation and the reference condition.

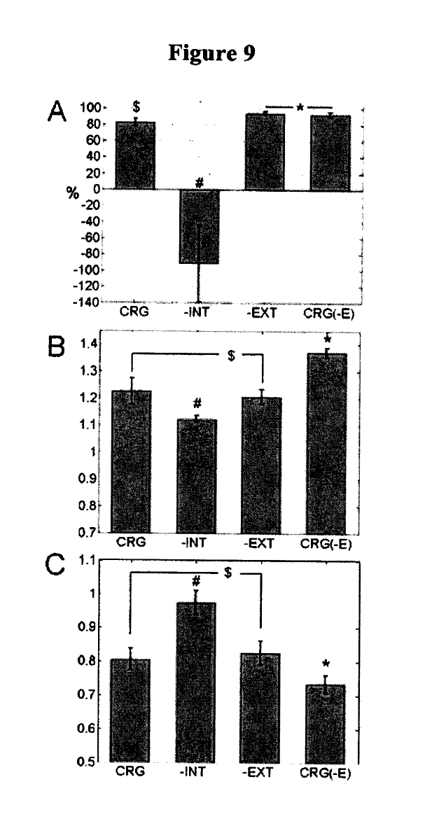

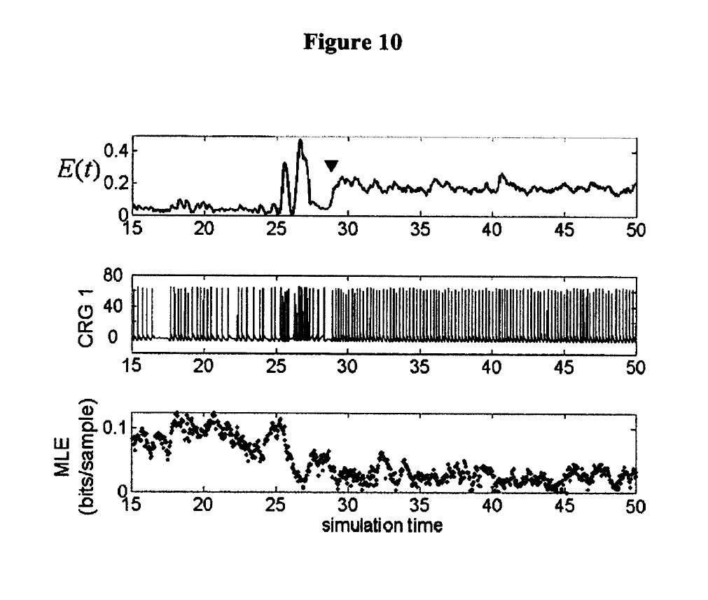

To examine the role of feedback in closed-loop CRG neuromodulation, external and internal feedback were independently disrupted and the effects on SLE suppression performance and complexity outcomes were noted (see FIG. 9). Eliminating internal feedback within the therapeutic network 12 amounted to de-tuning, and the result was a drastic change in the output dynamics of the therapeutic network 12, leading to a severe decline in performance. Abolishing the external feedback loop led to a slight decrease overall in temporal complexity of the epileptiform network dynamics and increase in mean phase coherence compared to the closed-loop configuration. What was unanticipated, however, was the apparent improvement in the SLE suppression achieved with the open-loop configuration. This unexpected result can be explained by looking at the stimulation-worsened runs of the closed-loop configuration. On infrequent occasions, while in closed-loop mode, the epileptiform network excitation level jumps to a pathological operating point near the threshold for seizure-like activity (see FIG. 10). Evidence of this pathological attractor is demarcated by the small secondary peak in the time series distribution of E(t) centered around 0.16 (see FIG. 6). The phenomenon was not observed to occur in open-loop configuration, suggesting that the more complex closed-loop case might be exhibiting some kind of bistable or multistable switching between high and low excitation, with the bias heavily in favour of the low excitation state(s). This was confirmed by examining SLE suppression performance and complexity, taking into account only the runs that excluded the high excitation level condition (but otherwise included both neuromodulation-improved and worsened runs). When high excitation level runs were excluded, the SLE suppression performance of the closed-loop configuration became statistically indistinguishable from the open-loop configuration. Moreover, the closed-loop MLE jumped to a value that far exceeded both the open-loop value and the previously-reported closed-loop value, indicating that the high excitation condition during closed-loop neuromodulation was indeed pathological and associated with lower dynamic complexity, acting as a drag on the MLE measure. Likewise, the mean phase coherence decreased to a value below the open-loop value and previous closed-loop value. Interpreted another way, the external feedback significantly improved target network complexity under normal closed-loop operation, compared to open-loop or non-responsive stimulation, but the presence of external feedback had the potential to push the network into a pathological mode of activity characterized by high excitation and low-complexity dynamics. This pathological closed-loop operation was observed to occur in only a handful of the total runs performed (˜12%), but was significant enough to impact the performance measures.

As will be appreciated from the above, the therapeutic network 12 is able to synthesize complex rhythmic stimulus waveforms for neuromodulation computationally using coupled CRGs. By tuning the parameters of the therapeutic network 12, a high-complexity stimulation signal possessing similar dynamic characteristics to electrographic recordings of neural activity was obtained. The multi-banded nature of the signal, characterized by high theta and gamma-frequency power and co-modulation of the theta and gamma bands, as well as the signal's dynamic complexity, quantified by a positive Lyapunov exponent and dimensionality comparable to biological neural signals, are attributable to the nonlinear interaction of coupled rhythm generators with different intrinsic properties and coupling.

Comparing the results of CRG neuromodulation to DBS-like LFS and HFS showed that the structural complexity of the stimulation was as important as the responsiveness of the stimulation in achieving high performance efficacy with respect to SLE suppression and boosting complexity of epileptiform network activity, and that those two aspects of stimulation were not mutually exclusive. Indeed, stimulation of the epileptiform network 14 using dynamically unresponsive periodic pulse stimulation did not fare as well as CRG neuromodulation. Even in open-loop configuration, with external feedback eliminated, CRG neuromodulation was able to reduce SLE occurrence by over 90%. However, when internal feedback was eliminated, SLE suppression performance was drastically reduced, while positive MLE values were still being generated in the target network, values that were higher than the unstimulated reference. This indicates the primary effect of the tuned therapeutic network stimulation in the absence of external feedback was suppression of SLEs. On the other hand, the inclusion of external feedback (i.e. closed-loop operation) was shown to significantly improve complexity of the epileptiform network, to an extent greater than open-loop operation, once the outliers involving pathological switching to high excitation level were excluded from the statistical analysis (see FIG. 9 and FIG. 10). This suggests that the feedback from the target biological system or network serves to modulate the therapeutic network's output dynamics according to the instantaneous state of the target biological system or network, in such a way that the responsiveness of neuromodulation is geared toward modifying the dynamic complexity of the stimulus signal, which in turn modulates the dynamic complexity of the target biological system or network.

Overall, HFS performed better than LFS when it came to SLE suppression, even though the neuromodulation-improved suppression rates for both frequencies of periodic pulse stimulation were comparable (˜35%). The fraction of neuromodulation-improved cases was larger for HFS than for LFS (64.2% vs. 45.8%, respectively). The advantage of HFS over LFS is likely attributable to its shorter pulse interval, which translates to a more frequent perturbation of the target network and hence more robust effect on network dynamics. Nevertheless, periodic pulse stimulation had a tendency to stabilize target network activity, resulting in levels of network entrainment that were comparable to or slightly worse than the reference (see FIG. 8), and yielding only modest improvements in temporal complexity due to SLE suppression. Therefore, while periodic stimulation reduced seizure occurrence, the stimulated network did not exhibit dynamics that could be considered normal. By contrast, biomimetic stimulation possessing a complex rhythmic structure effectively reduced entrainment and enhanced temporal complexity in the target biological system or network, suggesting that such an approach may achieve healthier dynamics in a biological system or network being treated with electrical stimulation.

Although embodiments have been described above with reference to the drawings, those of skill in the art will appreciate that variations and modifications may be made without departing from the scope thereof as defined by the appended claims.

### REFERENCES

Babloyantz A and Destexhe A 1986 Low-dimensional chaos in an instance of epilepsy Proc. Natl. Acad. Sci. U.S.A. 83 3513-7
Belluscio M A, Mizuseki K, Schmidt R, Kempter R and Buzsaki G 2012 Cross-frequency phase-phase coupling between theta and gamma oscillations in the hippocampus J. Neurosci. 32 423-35
Bergey G K and Franaszczuk P J 2001 Epileptic seizures are characterized by changing signal complexity Clin. Neurophysiol. 112 241-9
Bucher D, Prinz A A and Marder E 2005 Animal-to-animal variability in motor pattern production in adults and during growth J. Neurosci. 25 1611-9
Canolty R T, Edwards E, Dalal S S, Soltani M, Nagarajan S S, Kirsch H E, Berger M S, Barbaro N M and Knight R T 2006 High gamma power is phase-locked to theta oscillations in human neocortex Science 313 1626-8
Chiu A W, Jahromi S S, Khosravani H, Carlen P L and Bardakjian B L 2006 The effects of high-frequency oscillations in hippocampal electrical activities on the classification of epileptiform events using artificial neural networks J. Neural Eng. 3 9-20
Coenen A M L 1998 Neuronal phenomena associated with vigilance and consciousness: from cellular mechanisms to electroencephalographic patterns Conscious. Cogn. 7 42-53
Colic S, Zalay 0 C and Bardakjian B L 2011 Responsive neuromodulators based on artificial neural networks used to control seizure-like events in a computational model of epilepsy Int. J. Neural Syst. 21 367-83
Fell J, Roschke J and Beckmann P 1993 Deterministic chaos and the first positive Lyapunov exponent: a nonlinear analysis of the human electroencephalogram during sleep Biol. Cybern. 69 139-46
Fountas K N and Smith J R 2007 A novel closed-loop stimulation system in the control of focal, medically refractory epilepsy Acta Neurochir Suppl 97 357-62
Fountas K N, Smith J R, Murro A M, Politsky J, Park Y D and Jenkins P D 2005 Implantation of a closed-loop stimulation in the management of medically refractory focal epilepsy: a technical note Stereotact. Funct. Neurosurg. 83 153-8
Gear C W 1971 The automatic integration of ordinary differential equations Comm ACM 14 176-9 Gildenberg P L 2006 History of electrical neuromodulation for chronic pain. Pain Med. 7 S7-S13 Goaillard J M, Taylor A L, Schulz D J and Marder E 2009 Functional consequences of animal-to-animal variation in circuit parameters Nat. Neurosci. 12 1424-30
Grashow R, Brookings T and Marder E 2010 Compensation for variable intrinsic neuronal excitability by circuit-synaptic interactions J. Neurosci. 30 9145-56
Grassberger P and Procaccia I 1983 Measuring the strangeness of strange attractors Physica D 9 189-208 Hamani C, Andrade D, Hodaie M, Wennberg R and Lozano A 2009 Deep brain stimulation for the treatment of epilepsy Int. J. Neural Syst. 19 213-26
Hoke M, Lehnertz K, Pantev C and Latkenhoner B 1989 Spatiotemporal aspects of synergetic processes in the auditory cortex as revealed by magnetoencephalogram Series in Brain Dynamics ed E Basar and T H Bullock (Berlin: Springer)
Hooke R and Jeeves T A 1961 Direct search solution of numerical and statistical problems JACM 8 212-29
Iasemidis L D, Principe J C and Sackellares J C 2000 Measurement and quantification of spatiotemporal dynamics of human epileptic seizures Nonlinear Biomedical Signal Processing ed M Akay (Piscataway, N.J.: IEEE Press) pp 294-318
Jing H and Takigawa M 2000 Comparison of human ictal, interictal and normal non-linear component analyses Clin. Neurophysiol. 111 1282-92
Kang E E, Zalay OC, Cotic M, Carlen P L and Bardakjian B L 2010 Transformation of neuronal modes associated with low-Mg²⁺/high-K⁺ conditions in an in vitro model of epilepsy J Biol Phys 36 95-107
Kenney C, Simpson R, Hunter C, Ondo W, Almaguer M, Davidson A and Jankovic J 2007 Short-term and long-term safety of deep brain stimulation in the treatment of movement disorders J. Neurosurg. 106 621-5
Kossoff E H, Ritzl E K, Politsky J M, Murro A M, Smith J R, Duckrow R B, Spencer D D and Bergey G K 2004 Effect of an external responsive neurostimulator on seizures and electrographic discharges during subdural electrode monitoring Epilepsia 45 1560-7
Li X, Yao X, Fox J and Jefferys J G 2007 Interaction dynamics of neuronal oscillations analysed using wavelet transforms J. Neurosci. Methods 160 178-85
Loddenkemper T, Pan A, Neme S, Baker K B, Rezai A R, Dinner D S, Montgomery E B, Jr. and Luders H O 2001 Deep brain stimulation in epilepsy J. Clin. Neurophysiol. 18 514-32
Lozano A M 2001 Deep brain stimulation for Parkinson's disease Parkinsonism Relat Disord 7 199-203
Marder E and Goaillard J M 2006 Variability, compensation and homeostasis in neuron and network function Nat. Rev. Neurosci. 7 563-74
Mayberg H S, Lozano A M, Voon V, McNeely H E, Seminowicz D, Hamani C, Schwalb J M and Kennedy S H 2005 Deep brain stimulation for treatment-resistant depression Neuron 45 651-60
Mormann F, Lehnertz K, David P and Elger C E 2000 Mean phase coherence as a measure for phase synchronization and its application to the EEG of epilepsy patients Physica D 144 358-69
Nair S P, Shiau D S, Principe J C, Iasemidis L D, Pardalos P M, Norman W M, Carney P R, Kelly K M and Sackellares J C 2009 An investigation of EEG dynamics in an animal model of temporal lobe epilepsy using the maximum Lyapunov exponent Exp. Neurol. 216 115-21
Osorio I, Frei M G, Manly B F, Sunderam S, Bhavaraju N C and Wilkinson S B 2001 An introduction to contingent (closed-loop) brain electrical stimulation for seizure blockage, to ultra-short-term clinical trials, and to multidimensional statistical analysis of therapeutic efficacy J. Clin. Neurophysiol. 18 533-44
Osorio I, Frei M G, Sunderam S, Giftakis J, Bhavaraju N C, Schaffner S F and Wilkinson S B 2005Automated seizure abatement in humans using electrical stimulation Ann. Neurol. 57 258-68 Packard N, Crutchfield J, Farmer D and Shaw R 1980 Geometry from a time series Phys. Rev. Lett. 45 712-6
Peters T E, Bhavaraju N C, Frei M G and Osorio I 2001 Network system for automated seizure detection and contingent delivery of therapy J. Clin. Neurophysiol. 18 545-9
Popovic M R, Kapadia N, Zivanovic V, Furlan J C, Craven B C and McGillivray C 2011 Functional Electrical Stimulation Therapy of Voluntary Grasping Versus Only Conventional Rehabilitation for Patients With Subacute Incomplete Tetraplegia: A Randomized Clinical Trial Neurorehabil. Neural Repair
Roschke J, Fell J and Mann K 1997 Non-linear dynamics of alpha and theta rhythm: correlation dimensions and Lyapunov exponents from healthy subject's spontaneous EEG Int. J. Psychophysiol. 26 251-61
Rosenblum M, Pikovsky A, Kurths J, Schafer C and Tass P A 2001 Phase synchronization: from theory to data analysis Handbook of Biological Physics ed F Moss and S Gielen (Amsterdam: Elsevier Science B. V.) pp 279-321
Schreiber T and Schmitz A 2000 Surrogate time series Physica D 142 346-82
Serletis D, Zalay 0 C, Valiante T A, Bardakjian B L and Carlen P L 2011 Complexity in neuronal noise depends on network interconnectivity Ann. Biomed. Eng. 39 1768-78
Shadlen M N and Newsome W T 1998 The variable discharge of cortical neurons: implications for connectivity, computation, and information coding J. Neurosci. 18 3870-96
Sun F T, Morrell M J and Wharen R E, Jr. 2008 Responsive cortical stimulation for the treatment of epilepsy Neurotherapeutics 5 68-74
Theiler J 1987 Efficient algorithm for estimating the correlation dimension from a set of discrete points Phys. Rev. A 36 4456-62
Theiler J 1990 Estimating fractal dimension J. Opt. Soc. Am. A. 7 1055-73
Thrasher T A and Popovic M R 2008 Functional electrical stimulation of walking: function, exercise and rehabilitation Ann Readapt Med Phys 51 452-60
Thrasher T A, Zivanovic V, Mcllroy W and Popovic M R 2008 Rehabilitation of reaching and grasping function in severe hemiplegic patients using functional electrical stimulation therapy Neurorehabil. Neural Repair 22 706-14
Toda H, Hamani C and Lozano A M 2004 Deep brain stimulation in the treatment of dyskinesia and dystonia Neurosurg. Focus 17 E2
Wang X F, Chen G and Yu X 2000 Anticontrol of chaos in continuous-time systems via time-delay feedback Chaos 10 771-9
Wilson M and Bower J M 1992 Cortical oscillations and temporal interactions in a computer simulation of piriform cortex J. Neurophysiol. 67 981-95
Wyckhuys T, Boon P, Raedt R, Van Nieuwenhuyse B, Vonck K and Wadman W 2010 Suppression of hippocampal epileptic seizures in the kainate rat by Poisson distributed stimulation Epilepsia 51 2297-304
Young I T 1977 Proof without prejudice: use of the Kolmogorov-Smirnov test for the analysis of histograms from flow systems and other sources J. Histochem. Cytochem. 25 935-41
Zalay O C and Bardakjian B L 2008 Mapped clock oscillators as ring devices and their application to neuronal electrical rhythms IEEE Trans. Neural Syst. Rehabil. Eng. 16 233-44
Zalay 0 C and Bardakjian B L 2009 Theta phase precession and phase selectivity: a cognitive device description of neural coding J. Neural Eng. 6 036002
Zalay O C, Serletis D, Carlen P L and Bardakjian B L 2010 System characterization of neuronal excitability in hippocampus and its relevance to observed dynamics of spontaneous seizure-like transitions J. Neural Eng. 7 036002

The relevant portions of the references identified in the specification are incorporated herein by reference.

1. An apparatus for synthesizing a multi-band rhythmic signal comprising a therapeutic network having an input to receive an input signal representative of a seizure-like event, said therapeutic network being responsive to said input signal and configured to generate an output stimulation signal of a form to suppress said input signal.

2. The apparatus of claim 1 wherein the therapeutic network comprises an encoding stage in series with a decoding and generating stage.

3. The apparatus of claim 2 wherein the encoding stage is configured to process and transform the received input signal and the decoding and generating stage in response to encoding stage output generates the output stimulation signal, which can be applied to the system generating the input signal thereby to suppress the seizure-like event.

4. The apparatus of claim 3 wherein the encoding stage and the decoding and generating stage each comprise a feedback loop.

5. The apparatus of claim 4 wherein the feedback loop of the decoding and generating stage comprises a time delay.

6. The apparatus of claim 3 wherein the encoding stage and the decoding and generating stage each comprise a cognitive rhythm generator (CRG).

7. The apparatus of claim 6 wherein each CRG comprises a set or bank of neuronal modes whose mode outputs are combined by mixing functions, a ring device in the form of a limit-cycle oscillator, whose instantaneous amplitude and phase variables are modulated by the combined mode outputs and a mapper that maps the amplitude and phase variables to an observable output variable.

8. The apparatus of claim 1 wherein the input signal representative of the seizure-like event is generated by a biologic system.

9. The apparatus of claim 8 wherein the therapeutic network is configured to apply the stimulation signal to the biologic system to modify the generated input signal.

10. A method for suppressing a multi-band rhythmic signal comprising:
responsive to an input signal representative of a seizure-like event, generating, using a therapeutic network, an output stimulation signal of a form to suppress said input signal;
applying the stimulation signal to the input signal to generate a resultant signal;
feeding the resultant signal back to the therapeutic network as said input signal; and
repeating said generating, applying and feeding.

11. The method of claim 10 further comprising providing a time delay prior to said feeding.

12. The method of claim 10 wherein generating said output stimulation signal comprises generating at least two rhythm components of different frequency bands, the rhythm component of the lower frequency band modulating or coding the rhythm component of the higher frequency band.

13. The method of claim 10 further comprising receiving said input signal from a biologic system.

14. (canceled)

15. An apparatus for suppressing a multi-band rhythmic signal comprising a therapeutic network having an input configured to receive an input signal representative of a seizure-like event generated by a biologic system, said therapeutic network configured to generate an output stimulation signal of a form to suppress said input signal and apply the stimulation signal to the biologic system to modify the generated input signal, the modified input signal being fedback fed back to the input of said therapeutic network.

16. The method of claim 11 wherein generating said output stimulation signal comprises generating at least two rhythm components of different frequency bands, the rhythm component of the lower frequency band modulating or coding the rhythm component of the higher frequency band.

17. The apparatus of claim 1 wherein said output stimulation signal comprises at least two rhythm components of different frequency bands, the rhythm component of the lower frequency band modulating or coding the rhythm component of the higher frequency band.

18. The apparatus of claim 5 wherein said output stimulation signal comprises at least two rhythm components of different frequency bands, the rhythm component of the lower frequency band modulating or coding the rhythm component of the higher frequency band.

19. The apparatus of claim 6 wherein said output stimulation signal comprises at least two rhythm components of different frequency bands, the rhythm component of the lower frequency band modulating or coding the rhythm component of the higher frequency band.

20. The apparatus of claim 15 wherein said output stimulation signal comprises at least two rhythm components of different frequency bands, the rhythm component of the lower frequency band modulating or coding the rhythm component of the higher frequency band.
